(19) United States
(12) Patent Application Publication
(10) Pub. No.: US 2010/0010633 A1
(43) Pub. Date: Jan. 14, 2010

(54) Deployable Arc Fusion Cage and Methods Associated Therewith

(75) Inventor: Andrew Kohm, San Mateo, CA (US)

Correspondence Address: MEDTRONIC;Attn: Noreen Johnson - IP Legal Department, 2600 Sofamor Danek Drive, MEMPHIS, TN, 38132, US

(73) Assignee: Kyphon SARL, Neuchatel (CH)

(21) Appl. No.: 12/171,165

(22) Filed: Jul. 10, 2008

(51) Int. Cl. A61F 2/44

(52) U.S. Cl. 623/17.16; 623/17.15; 623/17.11

(57) ABSTRACT

Expandable fusion cages and methods associated therewith are disclosed. The present invention is generally directed to an apparatus and method for implanting an anterior installed intervertebral fusion cage system which can be selectively expanded between two adjacent vertebrae to cause them to change position relative to each other and produce a normal alignment of the spine, while promoting fusion of the vertebrae. Accordingly, the invention relates to an expandable fusion cage that may be inserted, in a reduced size configuration, into an intervertebral space and expanded after insertion to provide a desired size.

### FIELD OF THE INVENTION

The present invention relates to expandable fusion cages and methods associated therewith. Moreover, the present invention is generally directed to an apparatus and method for implanting an anterior installed intervertebral fusion cage system which can be selectively expanded between two adjacent vertebrae to cause them to change position relative to each other and produce a normal alignment of the spine, while promoting fusion of the vertebrae. The invention also relates to an expandable fusion cage that may be inserted, in a reduced size configuration, into an intervertebral space and expanded after insertion to provide a desired size.

### BACKGROUND OF THE INVENTION

Spinal implants can either be solid, sometimes referred to as a spacer or plug, or can define a hollow interior designed to permit bone in-growth, sometimes referred to as a fusion device or fusion cage. The interior of a fusion device may be filled with a bone growth inducing substance to facilitate or promote bone growth into and through the device. It is commonly accepted that spinal implants that facilitate or promote natural bone in-growth typically achieve a more rapid and stable arthrodesis. Some spinal implant designs are inserted into the disc space via a threading technique, while other designs are inserted into the disc space via a push-in or impaction technique.

Generally, fusion cages provide a space for inserting a bone graft between adjacent portions of bone. In time, the bone and bone graft grow together through or around the fusion cage to fuse the graft and the bone solidly together. One current use of fusion cages is to treat a variety of spinal disorders, including degenerative disc diseases, Grade I or II spondylolistheses, adult scoliosis and other disorders of the lumbar spine. Spinal fusion cages (included in the general term, “fusion cages”) are inserted into the intervertebral disc space between two vertebrae for fusing them together. They distract (or expand) a collapsed disc space between two vertebrae to stabilize the vertebrae by preventing them from moving relative to each other.

The typical fusion cage is cylindrical, hollow, and threaded. Alternatively, some known fusion cages are unthreaded or made in tapered, elliptical, or rectangular shapes. Known fusion cages are constructed from a variety of materials including titanium alloys, porous tantalum, other metals, allograft bone, carbon fiber or ceramic material.

Fusion cages may be used to connect any adjacent portions of bone, however one primary use is in the lumbar spine. Fusion cages can also be used in the cervical or thoracic spine. Fusion cages can be inserted in the lumbar spine using an anterior, posterior, or lateral approach. Insertion is usually accomplished through a traditional open operation, but a laparoscopic or percutaneous insertion technique can also be used.

With many of the approaches, threaded fusion cages are inserted by first opening the disc space between two vertebrae of the lumbar spine using a wedge or other device on a first side of the vertebrae. Next, a tapered plug is hammered in to hold the disc space open in the case of a threaded, cylindrical cage insert. A threaded opening is then drilled and tapped on a second side opposite the first side of the vertebrae for producing the equivalent of a “split” threaded bore defined by the walls of the vertebrae above and below the bore. The threaded fusion cage is then threaded into the bore and the wedge is removed. The first side is then drilled and tapped before inserting a second threaded fusion cage. Typically, two threaded fusion cages are used at each intervertebral disc level.

General techniques for inserting fusion cages are well known. Insertion techniques and additional details on the design of fusion cages is described in Internal Fixation and Fusion of the Lumbar Spine Using Threaded Interbody Cages, by Curtis A. Dickman, M. D., published in BNI Quarterly, Volume 13, No. 3, 1997, which is hereby incorporated by reference.

Traditionally anterior back surgery was done without much concern for the size of the incision. The large incisions allowed surgeons to deliver medical devices to the desired site without concern for the size of the medical device. However, with the development of small incision techniques (that ultimately result in faster healing times for patients) such as techniques like arthroscopic surgery, a premium has been placed upon being able to deliver medical devices through smaller incisions. In some instances, it is desired to be able to deliver one or more medical device(s) through a cannula, which when they arrive at the desired site, the medical device(s) can be expanded to a larger state that would not have fit through the cannula when the medical device is in its expanded state.

U.S. Pat. No. 5,782,832 to Larsen et al. (the “Larsen reference”) discloses a type of spinal fusion implant that one might associate with the more traditional back surgeries. FIG. 1 of the Larsen reference shows an implant apparatus with two separable support components which are adapted for adjusting sliding movement relative to each other to selectively vary the overall width of the implant to accommodate vertebral columns of various sizes or to vary the supporting capacity of the implant during healing. Each of the support components include upper and lower plate portions that are operatively connected by respective linkage mechanisms. The linkage mechanisms allow relative movement of the upper and lower plate portions between an extended position and a collapsed position. The device disclosed in the Larsen reference has several problems. One problem is that, because the width of the implant is adjusted prior to insertion, a wide insertion slot is necessary despite the reduced profile presented by the collapsed implant. Accordingly, insertion of the cage using non-invasive means such as through the use of a cannula tends to be somewhat limited. Another problem is that at least part of the linkage mechanism extends beyond the upper and lower plate portions, thus requiring more invasion into the body cavity to position the implant.

Brett, U.S. Pat. No. 6,126,689, illustrates an expandable and collapsible fusion cage, but its design uses hinges on two sides of the fusion cage, which makes its operation unwieldy. Moreover, the fusion cage can be difficult to expand when it is in the disk space, particularly in the instance wherein one desires non-invasive surgical techniques such as access to the spine using cannulas. Moreover, the fusion cage of Brett has only limited expanded state possibilities so that it is either in a fully expanded state or an intermediate partially expanded state.

Thus, what is desired is a expandable fusion cage that can easily be expanded when non-invasive surgical techniques are used (such as delivery through a cannula). The fusion cage should also be reliable and of a stable design that allows the insertable fusion cage to be incorporated and expanded by some type of inserting tool and/or an expansion tool which will allow the surgeon to implant the device without requiring extraordinary surgical skills (which arise from the design of the fusion cage).

### BRIEF SUMMARY OF THE INVENTION

The present invention relates to expandable fusion cages that can be inserted into adjacent vertebrae in an unexpanded state and can then be expanded. The fusion cages of the present invention are designed and configured so as to be readily inserted, accessible, and manipulated by non-invasive means. Moreover, in several of the embodiments of the present invention, the fusion cages are designed so as to allow the incorporation of other materials that will facilitate the insertion and manipulation of the device as well as the healing of the patient.

The present invention also relates to tools associated with the fusion cages of the present invention that allow the manipulation of the fusion cages once they have been put in their desired site.

### BRIEF DESCRIPTION OF THE SEVERAL VIEWS OF THE DRAWINGS

FIGS. 1A and 1B show a perspective view of the fusion cage with the deployable extensions (i.e., flexible elements) in their non-deployed state. FIG. 1C shows a cross sectional view of the fusion cage looking down the length of the fusion cage wherein the deployable extensions are in their non-deployed state.

FIGS. 2A and 2B show a perspective view of the fusion cage with the deployable extensions (i.e., flexible elements) in a semi-deployed state. FIG. 2C shows a cross sectional view of the fusion cage looking down the length of the fusion cage wherein the deployable extensions are in a semi deployed state.

FIGS. 3A and 3B show a perspective view of the fusion cage with the deployable extensions (i.e., flexible elements) in their fully deployed state. FIG. 3C shows a cross sectional view of the fusion cage looking down the length of the fusion cage wherein the deployable extensions are in a fully deployed state.

FIGS. 4A and 4B show two additional possible embodiments of the fusion cage that allow the deployable extensions (i.e., flexible elements) to be expanded (e.g., flexed in an outward direction).

FIG. 5 shows the fusion cage in one embodiment after it has been inserted between two vertebral bodies in its undeployed state.

FIG. 6 shows the fusion cage in one embodiment after it has been inserted between two vertebral bodies in its deployed state.

### DETAILED DESCRIPTION OF THE INVENTION

In an embodiment, the present invention relates to expandable fusion cages and methods associated therewith. The present invention generally relates to an apparatus and method for implanting an anterior installed intervertebral fusion cage system which can be selectively expanded between two adjacent vertebrae to cause them to change and hold their position relative to each other and produce a normal alignment of the spine, while promoting fusion of the vertebrae. The invention also relates to an expandable fusion cage that may be inserted, in a reduced size configuration, into an intervertebral space and expanded after insertion to provide a desired size.

An embodiment of the present invention relates to an expandable fusion cage comprising spacers and one or more deployable extensions (also called a “flexible element”), wherein the one or more deployable extensions are configured so that each deployable extension alternates with each spacer and the deployable extensions when deployed can extend or expand in a direction outside one or more planes of the spacers.

In an embodiment, the spacers can be rotating spacers (also referred to as a “rotating element”) and non-rotating spacers (also referred to as a “stationary element”). The rotating element may rotate relative to the stationary element causing the deployable element to be deployed so that it extends beyond at least one of an outer dimension of a stationary element and an outer dimension of a rotating element.

In an embodiment, the deployable extensions are deployed by rotating alternate spacers. When the alternate spacers are rotated, the deployable extensions, which are structurally attached (either directly or indirectly) to each of the spacers, the deployable extensions deploy so that they expand from a configuration that is inside one or more planes of the spacers to a configuration which is outside at least one plane of the spacers. In other words, they extend to a position beyond at least one of an outer dimension of a stationary element and an outer dimension of a rotating element.

In an embodiment, the deployable extensions are flexible in nature so that when a rotating element is rotated, a deployable extension (also referred to as a flexible element) transforms from a retracted configuration to an expanded configuration. This configuration change (from a smaller form to a larger form) can be achieved by having one end of the flexible element connected to the rotating spacer/element and having the other end of the flexible spacer connected to an adjacent stationary spacer/element. When rotation of the rotating spacer/element occurs, the flexible element flexes outward, thereby increasing the overall size of the fusion cage.

In an embodiment, the fusion cage, when it is in its non-deployed state, is of a size that allows the fusion cage to be delivered to the desired position between two vertebrae through a cannula. In a variation of this embodiment, the fusion cage can not pass through the same cannula when it is in its deployed/expanded state. In this variation, the fusion cage will be delivered to the desired site between two adjacent vertebrae in its unexpanded state wherein the extensions are then expanded to their expanded state. In one embodiment of this variation, a tool may be used that is configured to expand the deployable extensions while being deployed through the cannula. This tool or additional tools may be used to hold a terminal spacer and alternate spacers in a position that do not rotate.

In one embodiment, the fusion cage is configured to be rectangular in shape. Alternatively, the fusion cage may be cylindrical in shape. It should be understood that the shape of the fusion cage can be just about any shape that will allow the deployable extensions to provide the needed positioning and stabilizing of the vertebrae for the desired function (e.g., oval-, triangular-, horseshoe-, or star-shaped, among others). For example, the fusion cage should be able to provide support and structure for degenerative disc diseases, Grade I or II spondylolistheses, adult scoliosis and other disorders of the lumbar spine as well as for any other desired purpose.

When the fusion cage is rectangular in shape and when the deployable extensions are not deployed, the fusion cage has dimensions that are between about 0.6-1.0 cm in height, 0.6-1.0 cm in width and 2.0-5.0 cm in length. Alternatively, the fusion cage may have dimensions that are about 0.8 cm in height, 0.8 cm. in width and from 2.0 to 4.0 cm. in length. It has been found that fusion cages that are approximately these dimensions are suitable for placement between the vertebrae of a normal sized adult patient. However, it should be understood that other sizes are contemplated and therefore within the scope of the invention, for example, when the fusion cages are used on children or on adults that are of smaller or larger stature (e.g., for midgets or giants). The fusion cage may have the deployable extensions extend or expand beyond the planes of the spacers on two or more sides of the fusion cage.

When the deployable extensions are in a fully deployed state, the fusion cage may have dimensions that are between about 0.9-1.3 cm in height and/or 0.9-1.3 cm in width. Alternatively, the fusion cage may have dimensions that are about 1.1 cm in height and/or 1.1 cm in width. The height and/or width dimensions may remain the same as in the unexpanded state depending upon the one or more planes outside of which the deployed extensions are moved.

When the fusion cage is cylindrical in shape, it may have a radius of about 0.3-0.5 cm and a length of about 2.0-5.0 cm. In the expanded state the fusion cage may have a radius of between about 0.45 cm to about 0.65 cm. Again, as with the rectangular shaped fusion cage, these dimensions are ideally suited for the average size adult. It should be recognized that the dimensions may be modified so as to be ideally suited for use in children or for use in adults that are of smaller or larger stature.

The deployable extensions when deployed may be deployed in any of a plurality of dimensions and/or angular positions about the perimeter of the fusion cage. For example, in one variation the deployable extensions may all be deployed at a position that would be at 0 or 360 degrees about a longitudinal axis of the fusion cage (e.g., for a cylindrical fusion cage, an axis running through the circular cross-section of the cylinder. Alternatively, the deployable extensions when deployed may be extended at positions on opposite sides of the axis (i.e., the deployable extensions are extended at 0 degrees and at 180 degrees of the circle when viewed along the circular cross section of the cylinder). Alternatively, the deployable extensions may be deployed at three different angular positions about the axis (e.g., the deployable extensions would be deployed at positions that are at 0 degrees, 120 degrees and 240 degrees when the cylinder is viewed along a cross section). It should be understood from the above examples, that any of a plurality of deployments are possible for the deployable extensions including deployments at 4, 5 or more positions. Also, it should be apparent that the fusion cage may be designed so that the deployable extensions may be deployed at positions that are not equidistant around the cylinder (when viewed along the circular cross section). In addition, the deployable extensions can extend different distances away from the surfaces of the rotating elements. For example, the series of deployable extensions could exhibit increasing extension distances along the fusion cage, thereby creating a ramp-like configuration for the fusion cage when deployed. Alternatively, the central deployable extensions could exhibit lesser or greater extension lengths than the outer deployable extensions, thereby creating a “valley” or “mountain” configuration, respectively. Various other multi-extension-length configurations will be readily apparent.

In an embodiment, the deployable extensions may be deployed (extended) by rotating an inner pin that is configured to be parallel to the length of the fusion cage (not shown). In other words, the inner pin is positioned so that it is runs perpendicular to a circular cross section of a cylindrical fusion cage or perpendicular to the square cross section of a fusion cage that is for example, 0.8 cm in height, 0.8 cm in width and 3 cm in length. The inner pin may be positioned so that it is in the very center of the circle or square when viewed along the cross section of the cylindrical fusion cage or the square of the example of the above described fusion cage. The inner pin may be accessed at the terminal spacer of the fusion cage by a tool that is designed to pass through a cannula and is designed to rotate the inner pin, which in turn rotates the rotating element relative to the stationary element. In variations of this embodiment, the tool may be a modified screw driver or a modified hex driver. In a variation, one might have a handle that can be accessed at a proximal end of the cannula, the head of the screwdriver or hex driver is positioned so that it is at the distal end of the cannula. The head of the modified screwdriver or hex driver when being used through the cannula can be positioned so that it is either inside the cannula or positioned so that it extends beyond the distal end of the cannula. In this embodiment, it should be apparent that it is desired that the shaft of the screw driver or hex driver be approximately the same length or longer than the cannula that is to be used.

In an alternate embodiment, the deployment of the flexible element/deployable extension may occur via the rotation of one or more shafts to which the spacers and/or flexible elements are attached. In one embodiment, an inner shaft may be positioned within an outer shaft. One or more spacers (e.g., “inner spacers”) may be affixed to the inner shaft through openings (e.g., slots) in the outer shaft, one or more spacers (e.g., “outer spacers”) may be affixed to the outer shaft, and one or more flexible members may be connected between inner and outer spacers. Rotation of the inner shaft relative to the outer shaft would then result in rotation of the inner spacers relative to the outer spacers, which in turn would cause the flexible member(s) to flex outward to increase the overall size of the cage. In another embodiment, the inner and/or outer spacers could be eliminated, and the flexible member(s) could be attached directly to the inner and outer shafts. In another embodiment, the “relative rotation” behavior could be provided by adjacent (rather than concentric) shafts.

In an embodiment, the fusion cage is made of materials that are appropriate for being inserted in between vertebrae. For example, the spacers may be made of PEET (polyester/ether poly(ethylene-1,2-diphenoxyethane-p,p-dicarboxylate)), other hard polymeric plastics such as polyetheretherketone (PEEK) or a resorbable polymer such as polylactates (PLA), ceramic, or medical grade metallic materials such titanium. The deployable extensions are made of the same or different medical grade metallic material such as titanium or NITINOL (i.e., a nickel titanium alloy). It should be understood that other materials are contemplated such as stainless steel, silicone and metal materials, tantalum, platinum, titanium, and niobium alloys, PHYNOX®), or any of a plurality of polymeric materials, such as polytetrafluoroethylene (ePTFE), composite polymers, non-reinforced polymers, carbon-reinforced polymer composites, carbon fiber, PMMA, calcium hydroxide, ceramics, polylactide, polyglycolide, tyrosine-derived polycarbonate, polyanhydride, polyorthoester, polyphosphazene, calcium phosphate, calcium hydroxide, hydroxyapatite, bioactive glass, or any combination of these or other materials. In all cases, the selected material for a part should be able to be use for the intended purpose of that part.

In an embodiment of the present invention, the material that may be used may ultimately be resorbed into the patient so that only bone that has undergone accretion is present between the adjacent vertebrae.

Other materials/chemicals may be associated with the fusion cage of the present invention such as lubricants, antibiotics, anti-cancer agents, bone cements, grafts, chemicals that prevent autoimmune defenses, and the like. In an embodiment, these materials/chemicals may be released in a time lapse fashion. These materials/chemicals may be used to aid accretion of bone or of tissue, to aid in fighting off bacterial infections, to promote tissue growth, to prevent or promote sliding of the fusion cage, or for a plurality of other reasons.

Chemical compositions may be associated with the fusion cage such as those disclosed in US Patent Application Publication No. 20060240064 to Hunter (which is herein incorporated by reference in its entirety), which may prevent the fusion cage from moving once it has been placed in its desired position.

Thus, in an embodiment, the present invention is also directed to kits, which in addition to the fusion cage, may optionally contain one or more of the appropriate cannulae, other tools, chemicals (or other materials like bone cements), as well as written instructions and other materials that would appropriately belong in kits.

In an embodiment, the present invention is also related to a method of positioning and stabilizing adjacent vertebrae comprising the steps of

inserting an expandable fusion cage between the adjacent vertebrae wherein the expandable fusion cage comprises spacers and one or more deployable extensions and each deployable extension alternates with each spacer,

deploying the one or more deployable extensions so that the deployable extensions extend or expand in a direction outside a plane of the spacers thereby positioning and stabilizing the adjacent vertebrae.

Additional method steps may be present such as placing the fusion cage at the desired site by use of a cannula. After the fusion cage is at the desired site, the fusion cage may be accessed by tools through the cannula. These tools may be tools such as an extended screw driver or a hex wrench or some other tool that allows alternate spacers on the fusion cage to be rotated, which in turn deploys the deployable extensions. A possible method step is accessing the inner pin with a tool through a cannula, rotating the inner pin, which rotates alternating spacers, which in turn deploys the deployable extensions.

The methods of the present invention allow the treatment of any vertebrae, for example, the fusion cage can be applied to pelvic, lumbar, thoracic, or cervical vertebrae.

With reference to the figures, the below describes the fusion cage and methods associated therewith more specifically. This description is in no way meant to limit the scope of the present invention but is only meant to be illustrative of particular species that illustrate the workings of the present invention.

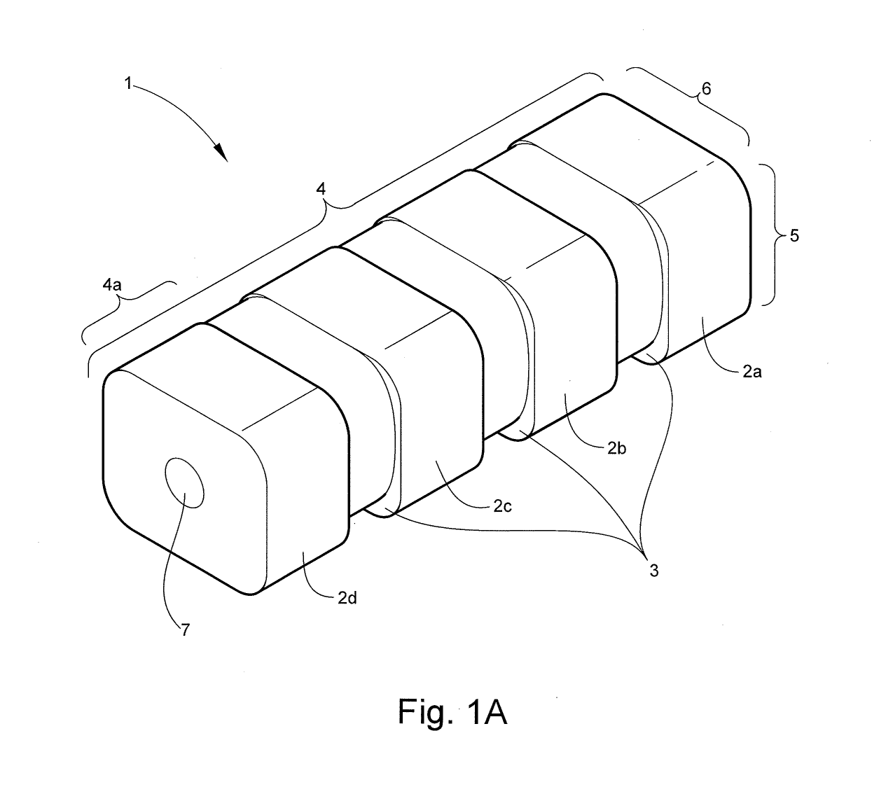

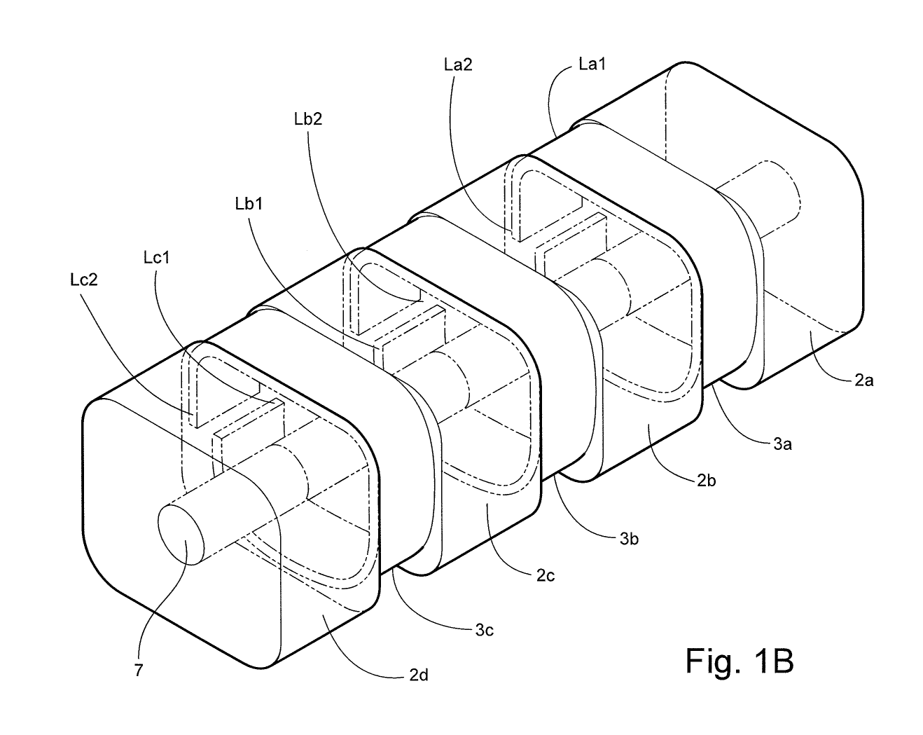

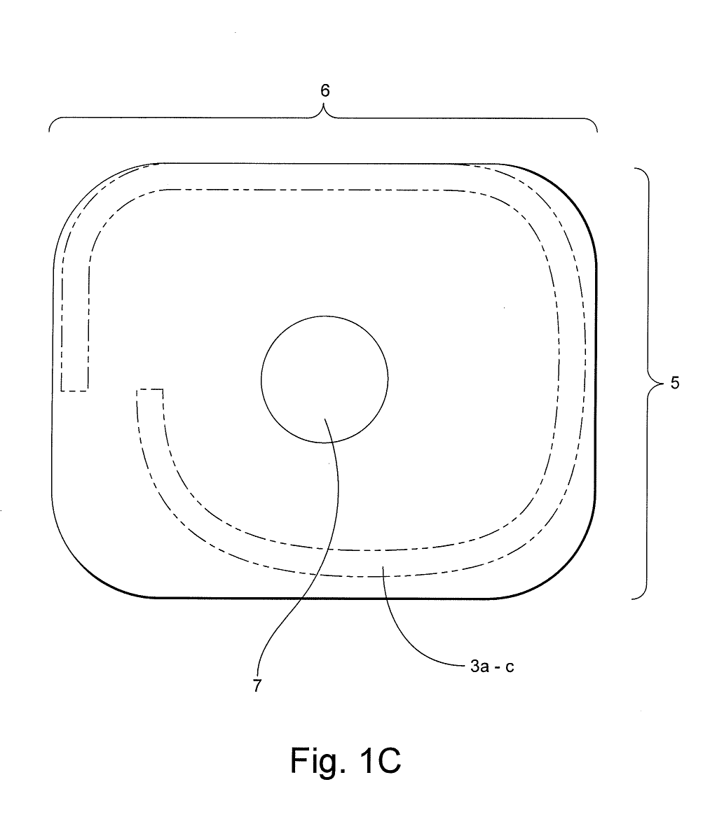

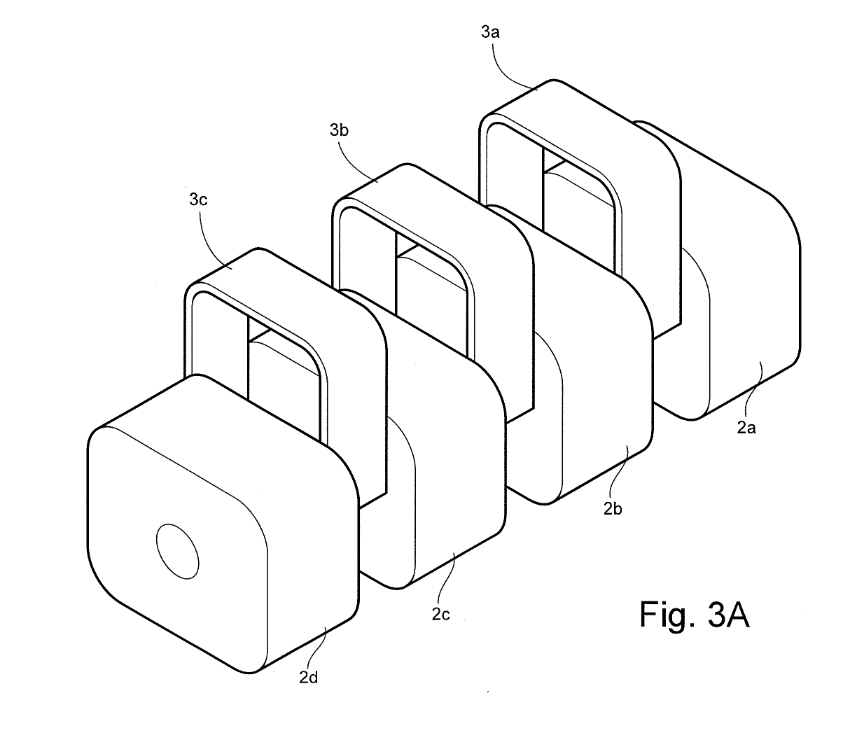

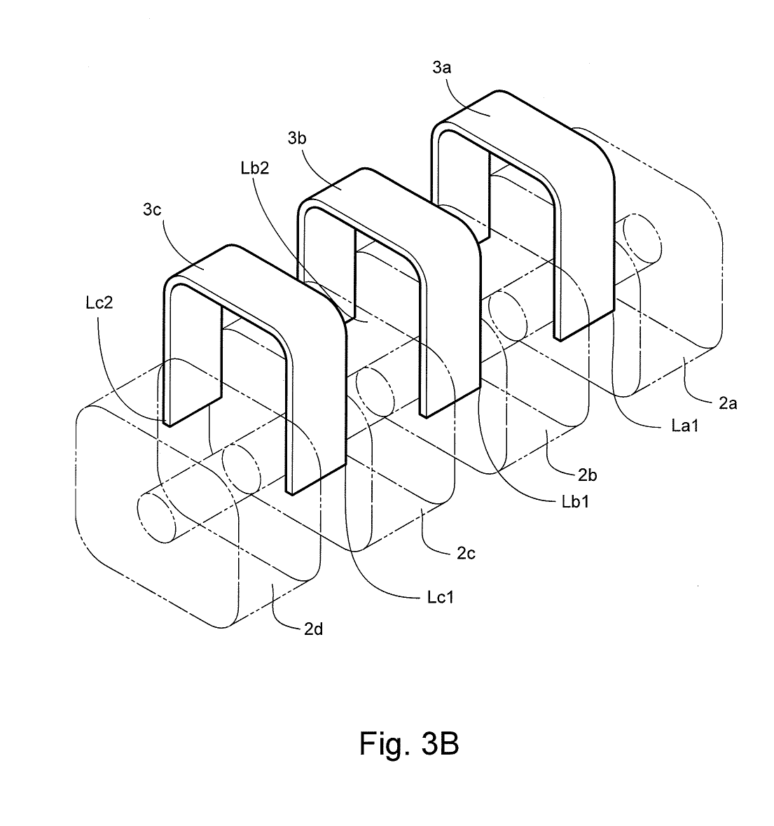

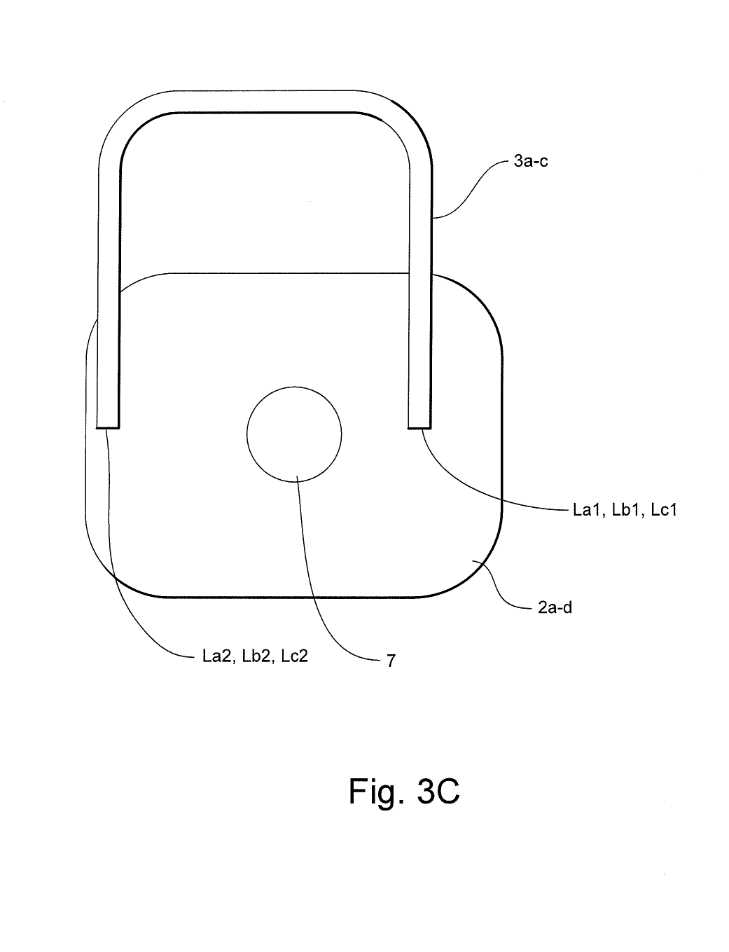

FIG. 1A illustrates a perspective view of the fusion cage 1 which shows both spacers 2a, 2b, 2c, 2d and deployable extensions/flexible elements 3. Spacer 2a is the terminal spacer on the proximal side of the fusion cage and spacer 2d is the terminal spacer on the distal side of the fusion cage. Although this fusion cage is shown with four spacers and three deployable extensions, it should be understood that any of a plurality of spacers and deployable extensions can be used in the present invention. Accordingly, if more spacers are present, the spacers would tend to be thinner in length 4a whereas if fewer spacers are present, the spacers can be thicker in length. In a variation, the total length 4 of the fusion cage is between about 2.0 and 5.0 cm. The fusion cage 1 as shown in this embodiment is rectangular in shape and the deployable extensions 3, when deployed transform from a retracted configuration in which the deployable extensions 3 remain inside or even with the planes of the spacers to an expanded configuration in which the deployable extensions 3 extend outside of the planes of the spacers (formed by the length of the spacers 5 and the width of the spacers 6—hereinafter referred to as being inside or outside the fusion cage). The deployment of the deployable extensions can better be seen in FIGS. 3A-C. It should be noted that although this embodiment is shown as being rectangular, it should be understood that the present invention also encompasses a cylindrical design and the appropriate modifications can be made to accommodate the cylindrical design.

FIG. 1B shows a perspective view of the fusion cage wherein the spacers are transparent so one can better see the interconnections between deployable extensions 3 and spacers 2a-2d. One end of deployable extension 3a is attached to spacer 2a at a location La1, and the other end of deployable extension 3a is attached to spacer 2b at a location La2. One end of deployable extension 3b is attached to spacer 2c at a location Lb1 and the other end of deployable extension 3b is attached to spacer 2b at a location Lb2. Finally, one end of deployable extension 3c is attached to spacer 2c at a location Lc1 and the other end of deployable extension 3c is attached to spacer 2d at a location Lc2.

FIG. 1C shows the fusion cage looking at it along a cross section of the length. Note that in this figure, the width 6 of the fusion cage is slightly larger than the height 5 of the fusion cage (although it should be understood that the width may be less than the height or alternatively, the width and the height may be of the same dimensions so that a cross sectional view of the fusion cage along its length is a square). FIG. 1C depicts the retracted configuration of flexible elements 3a-3c, in which those flexible elements are essentially “wound” around the longitudinal axis of cage 1 (in this case a central element 7).

To deploy flexible elements 3a-3c, some of the spacers are rotated relative to the other spacers. The rotating spacers may be referred to herein as “rotating elements” and the stationary spacers may be referred to herein as “stationary elements”. In the specific embodiment described with respect to FIGS. 1A-C, 2A-C, and 3A-C, spacers 2a and 2c can be considered the rotating elements, and spacers 2b and 2d can be considered the stationary elements.

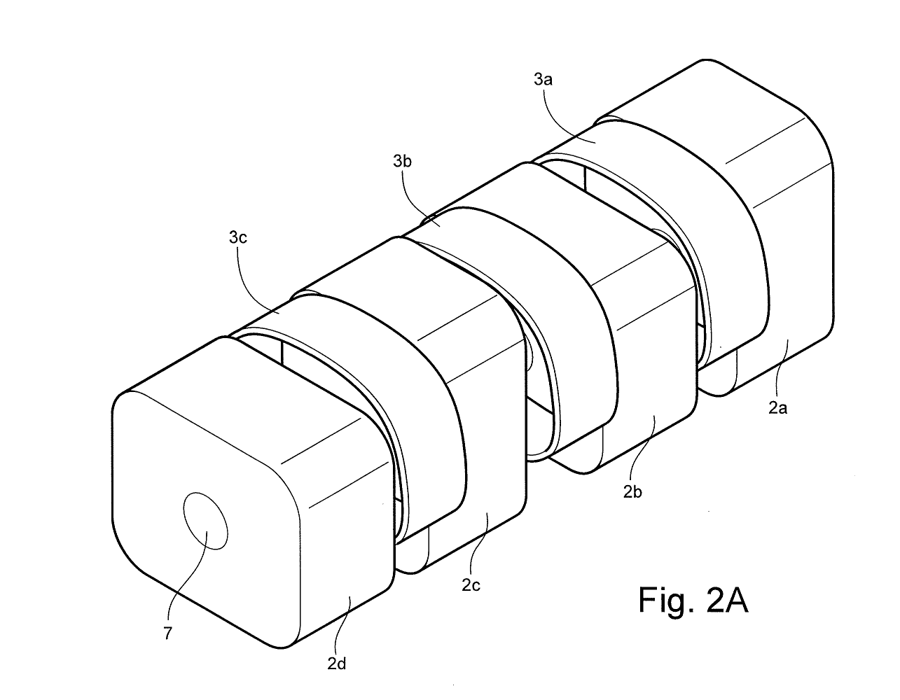

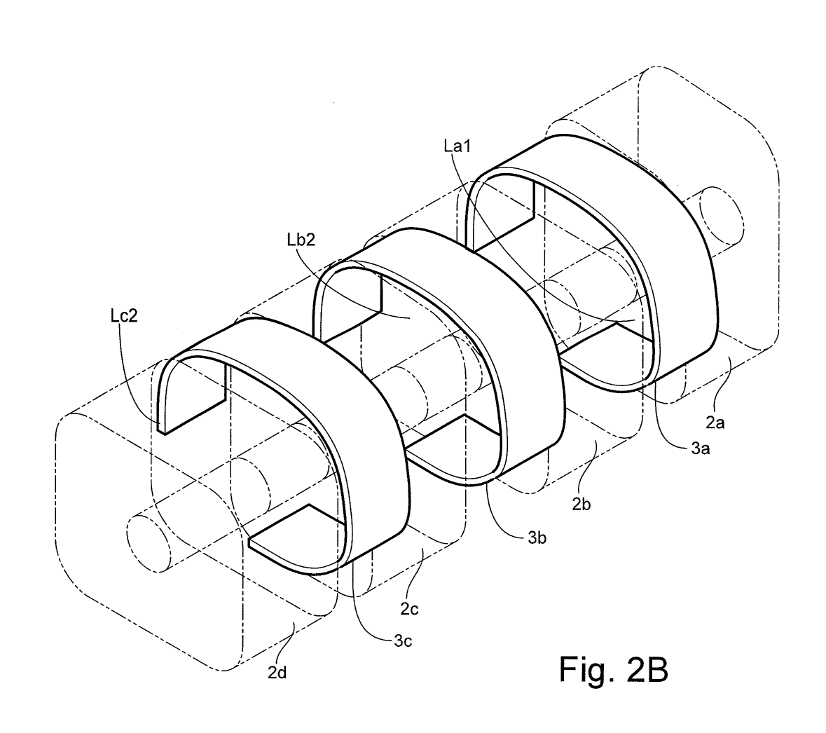

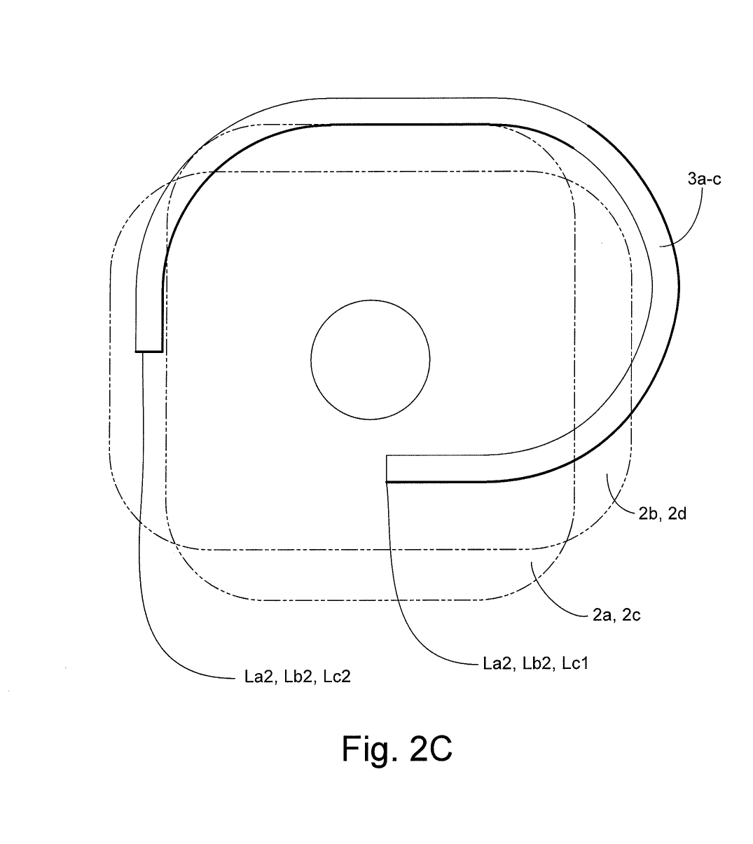

FIGS. 2A-2C show flexible elements 3a-3c being partially deployed via the rotation of spacers 2a and 2c relative to spacers 2b and 2d (in this case a 90° relative rotation). Specifically, because each flexible element 3a-3c has one end connected to a stationary element and one end connected to a rotating element, as spacers 2a and 2c rotate relative to spacers 2b and 2d, flexible elements 3a-3c begin to “unwind” from around the longitudinal axis of cage 1. As can be seen in FIG. 2C, this unwinding occurs due to the fact that one end of each flexible element 3a-3c (e.g., the end of flexible element 3c attached to spacer 2c at location Lc1) is rotated around an axis of rotation (e.g., central element 7) of the rotating element(s) relative to the other end of the flexible element (e.g., the end of flexible element 3c attached to spacer 2d at location Lc2). As can be further seen in FIG. 2C, the unwinding causes flexible elements 3a-3c to begin flexing outward into their expanded configuration.

Further rotation of spacers 2a and 2c relative to spacers 2b and 2d (in this case a 180° relative rotation) results in the fully expanded configuration of flexible elements 3a-3c, as shown in FIGS. 3A-3C As can be seen in FIG. 3C, the overall height of cage 1 has been significantly increased due to the deployment of flexible elements 3a-3c. Note that the rotation of the rotating elements (e.g., spacers 2a and 2c) relative to the stationary elements (e.g., 2b and 2d) can be varied to adjust the overall size of cage 1. For example, spacers 2a and 2c could be rotated 120° relative to spacers 2b and 2d to provide a cage height less than that depicted in FIG. 3C.

Various mechanisms can be used to provide the relative rotation capability of the rotating elements (e.g., spacers 2a and 2c) and the stationary elements (e.g., spacers 2b and 2d) in cage 1. In one embodiment, the rotating elements can be attached to a first shaft and the stationary elements can be attached to a second shaft, wherein the first and second shafts can rotate with respect to each other.

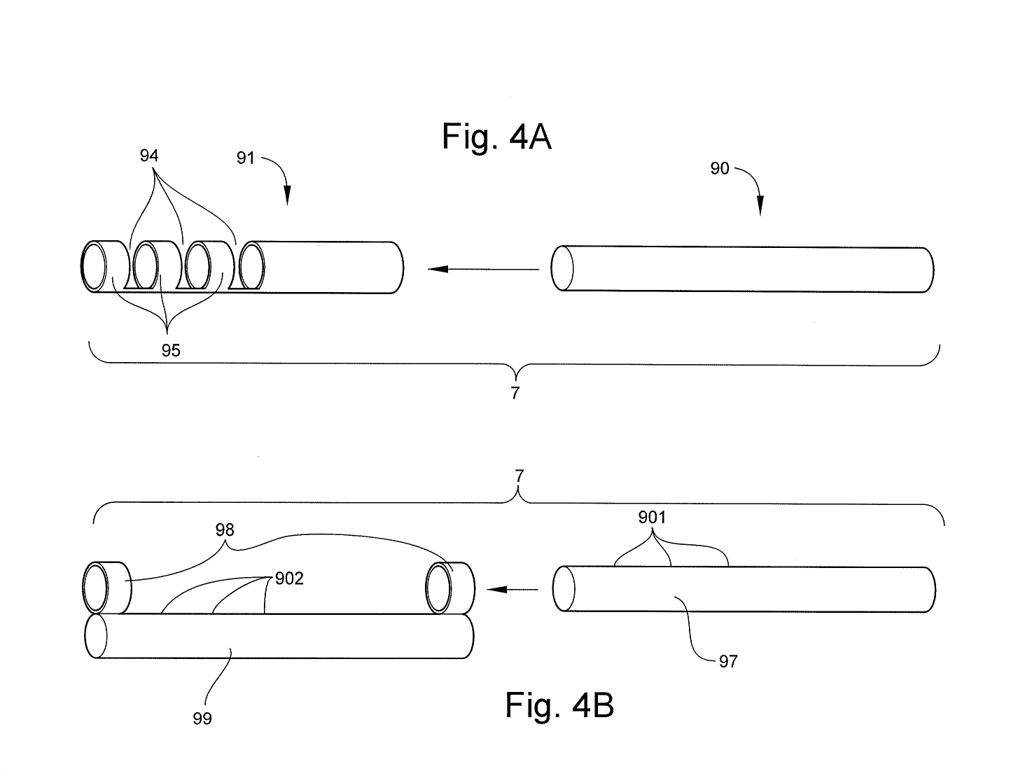

For example, FIG. 4A shows an embodiment of central element 7 (shown in FIGS. 1A-1C, 2A-2C, and 3A-3C) that includes an outer shaft 91 and an inner shaft 90 (shown disassembled for clarity in FIG. 4A). Outer shaft 91 includes one or more openings 94 through which rotating elements (e.g., spacers 2a and 2c) can be attached to inner shaft 90. Meanwhile, stationary elements (e.g., spacers 2b and 2d) can be attached to attachment portions 95 adjacent to openings 94. Rotating inner shaft 90 relative to outer shaft 91 would therefore cause the rotating elements to rotate relative to the stationary elements.

FIG. 4B shows another embodiment of central element 7 that includes a stationary shaft 99 and a rotating shaft 97 (shown disassembled for clarity). Stationary shaft 99 includes bushings 98 through which rotating shaft 97 is installed, thereby allowing rotating shaft 97 to rotate independently of stationary shaft 99. Rotating elements (e.g., spacers 2a and 2c) can be attached to attachment locations 901 on rotating shaft 97, and stationary elements (e.g., spacers 2b and 2d) can be attached to attachment locations 902 on stationary shaft 99. Rotating shaft 97 relative to stationary shaft 99 would therefore cause the rotating elements to rotate relative to the stationary elements. Note that the rotating elements would require an internal opening large enough to allow those elements to rotate about stationary shaft 97, just as the stationary elements would require an internal opening large enough to allow rotating shaft 99 to rotate.

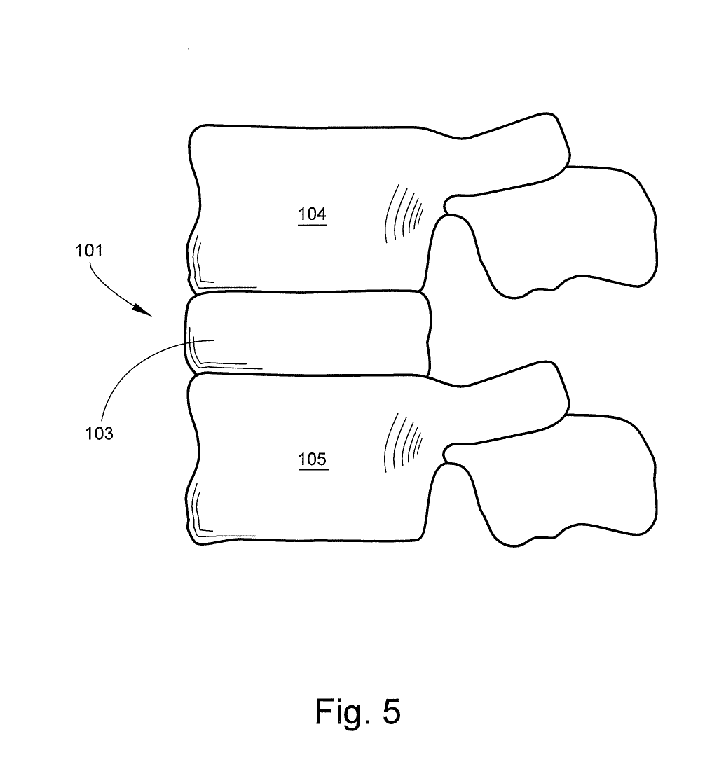

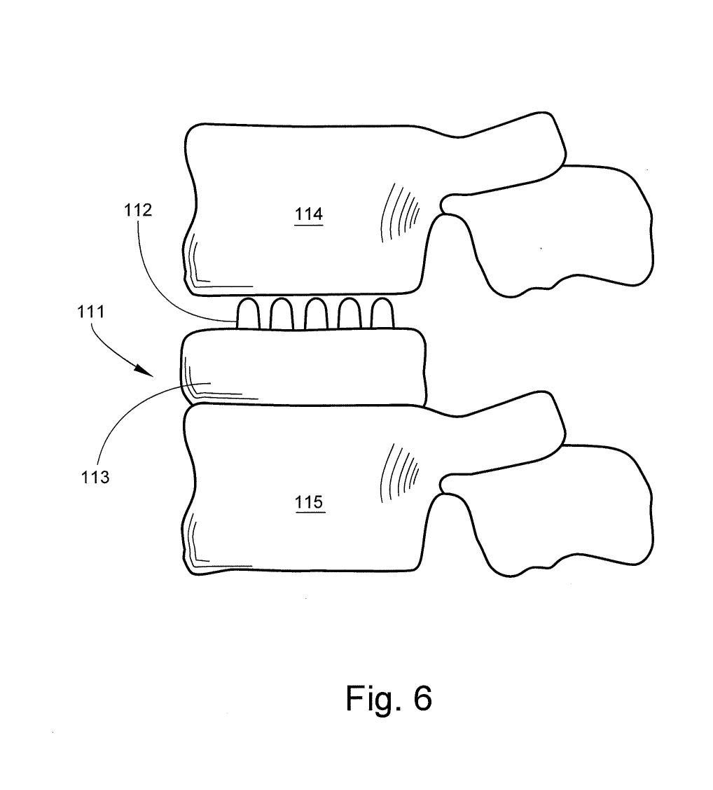

FIG. 5 shows an embodiment of a fusion cage 101 that has been inserted between two vertebrae 104, 105 wherein the flexible elements/deployable extensions (not shown in the figure) have not been deployed. The vertebrae in this embodiment may not be properly anatomically spaced. For example, the patient into which this fusion cage 101 has been placed may have a spinal medical problem that the patient is having alleviated by the insertion of the fusion cage (contrast with FIG. 6).

As shown in FIG. 6, when the rotating spacer(s) are rotated relative to the stationary spacer(s) (all shown generically as 113 in FIG. 11), the flexible elements 112 position vertebrae 114, 115 into their correct anatomical position. Once the vertebrae have attained their correct anatomical position, the surgeon has the option of adding bone cement (or some other material) through cannula (not shown) to facilitate fusion of the vertebrae. Alternatively, the bone cement (or other material) may have been associated with fusion cage 111 when initially inserted into the space between the vertebrae (as shown in FIG. 5).

It should be understood that the scope of the present invention encompasses any one or more described element or embodiment combined with any one or more described element or embodiment. Moreover, when ranges are disclosed above, it should be understood that any value that fits within the disclosed range is contemplated as a potential end point for any sub-range. Accordingly, these possible end point values are therefore within the scope of the present invention. For example, if a range of 0.9 to 1.3 is stated, it should be understood that a sub-range of 1.08 to 1.24 is contemplated and therefore within the scope of the instant invention, even if those values are not explicitly recited in the written description.

With this description of the embodiments and illustrated by the figures of the present invention, it should be apparent that modifications can be made to the above described embodiments without departing from the spirit and scope of the invention. The above written description provides a description of the structure and methods of use of exemplary embodiments of the invention. However, the above discussed variations and embodiments are to be construed so that the invention contains other reasonable variations of the invention that are not explicitly described. In any event, the invention is to be defined by the appended claims.

We claim:

1. An expandable fusion cage comprising:
a first stationary element;
a first rotating element; and
a first flexible element,
wherein the first flexible element is connected between the first stationary element and the first rotating element, and
wherein the first flexible element is configured to flex outward when the first rotating element is rotated in a first direction relative to the first stationary element.

2. The fusion cage of claim 1, wherein the fusion cage is configured to be rectangular in shape.

3. The fusion cage of claim 1, wherein the fusion cage is configured to be cylindrical in shape.

4. The fusion cage of claim 1, further comprising a plurality of flexible elements.

5. The fusion cage of claim 4, wherein the plurality of flexible elements are made of titanium or NITINOL.

6. The fusion cage of claim 1, wherein the first flexible element is fully deployed when the first stationary element is rotated 180 degrees relative to the first rotating element. 7. The fusion cage of claim 1, further comprising a cannula and a tool that is configured to rotate the rotating element relative to the stationary element resulting in the outward flex of the flexible element while the cage is deployed through the cannula

8. The fusion cage of claim 4, wherein the fusion cage comprises at least two stationary elements and at least two rotating elements.

9. The fusion cage of claim 8, further comprising in inner pin wherein the inner pin is structurally connected to the rotating elements so that when the inner pin is rotated each of the rotating elements rotate relative to the other stationary elements which flexes the first flexible element in an outward direction.

10. The fusion cage of claim 9, wherein the inner pin is structurally connected to the rotating elements by a gear and cog design.

11. The fusion cage of claim 1, wherein the first stationary element and the first rotating element are made of polyetheretherketones or titanium and the first flexible element is made of titanium or NITINOL.

12. An expandable fusion cage comprising:
a first stationary element;
a first rotating element; and
a first flexible element,
wherein a first end of the first flexible element is connected to the first rotating element,
wherein a second end of the first flexible element is connected to the first stationary element, and
wherein the first rotating element is configured to rotate independently from the first stationary element, and
wherein the first rotating element is configured to rotate the first end of the first flexible element relative to the second end of the first flexible element about a first axis of rotation.

13. The fusion cage of claim 12, further comprising
a second stationary element;
and a second flexible element;
wherein the second flexible element is positioned between the second stationary element and the first rotating element and the second flexible element is connected to the first rotating element the second stationary element, and
wherein when the first rotating element is rotated relative to the second stationary element the second flexible element extends beyond at least one of the outer dimension of the first stationary element, an outer dimension of the second stationary element and the outer dimension of the first rotating element.

14. The fusion cage of claim 13, wherein the first rotating element rotates up to 180 degrees relative to the first stationary element or second stationary element.

15. The fusion cage of claim 13, further comprising a bone cement or a bone graft.

16. The fusion cage of claim 13, wherein the stationary and rotating elements are made of polyetheretherketones or titanium and the flexible elements are made of titanium or NITINOL.

17. The fusion cage of claim 13, wherein the fusion cage prior to the first rotating element being rotated is about 2.0 to 5.0 cm in length, about 0.6 to 1.0 cm in height and about 0.6 to 1.0 cm in width.

18. A method of positioning and stabilizing adjacent vertebrae comprising
inserting an expandable fusion cage between the adjacent vertebrae wherein the expandable fusion cage comprises
a first stationary element;
a first rotating element; and
a first flexible element,
wherein the first flexible element is connected between the first stationary element and the first rotating element, and
wherein the first flexible element is configured to flex outward when the first rotating element is rotated in a first direction relative to the first stationary element
thereby positioning and stabilizing adjacent vertebrae.

19. The method of claim 18, wherein the fusion cage is configured to be rectangular or cylindrical in shape.

20. The method of claim 19, further comprising a plurality of flexible elements 21. The method of claim 20, wherein the adjacent vertebrae are accessed with the fusion cage by a cannula.

22. The method of claim 21, further comprising inserting a bone cement or a bone graft.

23. The method of claim 22, wherein the vertebrae are lumbar vertebrae.

24. A kit comprising a fusion cage, a cannula and written instructions, wherein the fusion cage comprises:
a first stationary element;
a first rotating element; and
a first flexible element,
wherein the first flexible element is connected between the first stationary element and the first rotating element, and
wherein the first flexible element is configured to flex outward when the first rotating element is rotated in a first direction relative to the first stationary element.

25. The kit of claim 24, further comprising a tool that allows the fusion cage to be deployed.
